(19) United States
(12) Patent Application Publication
(10) Pub. No.: US 2026/0283689 A1
(43) Pub. Date: Sep. 24, 2026

(54) LASER THERAPY SYSTEM FOR SELECTING AND USING OPTICAL DATA

(71) Applicant: GYRUS ACMI, INC. D/B/A OLYMPUS SURGICAL TECHNOLOGIES AMERICA, Westborough, MA (US)

(72) Inventors: Kurt G. Shelton, Bedford, MA (US); Sergey A. Bukesov, Acton, MA (US); Yi-Chia Lin, Lexington, MA (US)

(21) Appl. No.: 19/473,972

(22) PCT Filed: Apr. 2, 2024
(86) PCT No.: PCT/US2024/022659

§ 371 Date: Oct. 9, 2025

Related U.S. Application Data
(60) Provisional application No. 63/495,226, filed on Apr. 10, 2023.

(51) Int. Cl. A61B 18/22; A61B 18/00; A61B 18/20

(52) U.S. Cl. CPC A61B 18/22; A61B 2018/00642; A61B 2018/00708; A61B 2018/00773; A61B 2018/2015

(57) ABSTRACT

A system for selecting and using optical data obtained from a target at a target location of a patient may include an 2024/215512 optical detector. The optical detector may be configured to collect optical data from the target location. The system may also include processor circuitry. The processor circuitry may be configured to receive the optical data from the optical detector and may use at least one criterion to select data from the optical data based at least in part on whether a therapy light beam is operating. The processor circuitry may determine a characteristic of the target based on the selected data from the optical data.

WO

### PRIORITY CLAIM

This application claims the benefit of priority to U.S. Provisional Patent Application Ser. No. 63/495,226, filed Apr. 10, 2023, the contents of which are incorporated herein by reference.

### TECHNOLOGICAL FIELD

The present disclosure relates to optical systems, and more particularly, but not by way of limitation, to a system for collecting information about a target using an optical sensor.

### BACKGROUND

Modern systems can use optical detectors to measure electromagnetic waves. Optical detectors may generate optical data corresponding to at least a portion of the measured electromagnetic waves. Examples of such modern systems include light intensity sensors, cameras, and spectrometers. The collected optical data may be processed to determine a characteristic of the source of the optical data.

### SUMMARY

Laser or plasma systems may be used for delivering therapeutic laser energy to various target treatment areas such as may include soft or hard tissue. Examples of laser therapy may include ablation, coagulation, vaporization, fragmentation, etc. In lithotripsy applications, laser therapy may be used to break down calculi structures in one or more of the kidney, gallbladder, ureter, or other stone-forming regions, or to ablate large calculi into smaller fragments.

Endoscopes may be used to provide access to an internal location of a patient such that a physician may be provided with visual access. An endoscope may be inserted into a patient's body, deliver light to a target (e.g., a target anatomy or object) being examined, and collect light reflected, scattered, or otherwise emitted from the target. The reflected light may carry information about the target being examined. Endoscopes may include a working channel through which the operator can perform suction or pass instruments, such as may include a laser fiber, a stone-removal basket, brushes, biopsy needles, or forceps, or perform minimally invasive surgery to remove unwanted tissue or foreign objects from the patient.

In certain procedures employing electromagnetic energy, such as may include laser or plasma, it may be difficult to identify the composition of a target while executing the procedure. For health-related procedures, it may be difficult for a physician to identify whether a target is soft or hard tissue in vivo. In an approach, a surgical method may be used to extract tissue or other material and then identify the composition of the tissue or other material once it has been removed from the body. This approach may be one or more of time-consuming, potentially damaging to the patient, or difficult to perform. Techniques that use an extracted sample of the target to attempt to identify the composition of a target may not be able to monitor the composition of the target on an ongoing basis through all or a portion of a procedure.

The present inventors have recognized, among other things, that an optical detector (such as a light intensity sensor, a camera, or spectrometer) may be used to collect information about the target in real time during a procedure as opposed to analyzing a target sample after it is removed from the site. Light reflected or otherwise returned or emitted from a target may be analyzed, such as may include using spectroscopy, such as to detect and characterize the target, to differentiate the target from tissue, or the like. This may allow measurement or analysis of the composition, shape and/or size of an anatomical target, such as may be at the tip of the endoscope or laparoscope. The information obtained from the target may be used to adapt a treatment method during the procedure. For example, if a procedure involves breaking a renal calculus that has a hard surface, but a soft core, into tiny pieces (e.g., “dusting” the renal calculi) the recurrent or other ongoing monitoring of the target composition via the endoscope or laparoscope using approaches described herein can allow for adjustment of the procedure in real time. For example, the settings of the instrument used to break the calculus can be adjusted during the procedure. For example, this can permit adjusting one or more laser settings for a laser ablation instrument, such as during the procedure. The identification of the target composition or other target characteristic can allow for first providing settings that perform better on the hard surface of the stone to then providing settings that perform better on the soft core of the stone.

A therapeutic laser, such as may be used during a surgical laser procedure, may affect the optical data collected from the target. This may be due to flashing, vapor bubbles, or fiber vibration. An aiming beam, such as a visible laser, may be used to determine where the therapeutic laser is directed. The aiming beam may affect the optical data received from the target. Therefore, the present inventors have recognized, among other things, that it may be desirable to analyze optical data that was collected when one or more of the therapeutic laser or the aiming beam are in a desired state, such as may include an “off” state during which the therapeutic laser beam and/or aiming beam are not operating. In an approach, the collection of optical data may be sequenced based upon a state of a beam. This may require synchronization between two or more systems, such as may include an optical signal collection/detection system and a beam generation system. This may cause an additional load on a processor performing the sequencing or synchronization. The sequencing may be inaccurate, such as may be due to uncertainty surrounding the operation of one or more beams. The present inventors have recognized, among other things, that it may be desirable to collect data initially, and then select desired data for further analysis, such as may obviate the need for synchronization of the systems. This document describes, among other things, a system for selecting optical data that is collected when one or more beams are in a desired operating state.

In an example, a system for selecting and using optical data obtained from a target at a target location of a patient may include an optical detector. The optical detector may be configured to collect optical data from the target location. The system may also include processor circuitry. The processor circuitry may be configured to receive the optical data from the optical detector and may use at least one criterion to select data from the optical data based at least in part on whether a therapy light beam is operating. The processor circuitry may determine a characteristic of the target based on the selected data from the optical data.

In an example, a computer-implemented method of operating a system for selecting optical data obtained from a target at a target location of a patient may include using an optical detector, collecting optical data corresponding to the target location. The method may also include determining whether at least one of a therapy light beam or a secondary light beam is operating during the collecting optical data. The method may also include using processor circuitry, selecting data from the optical data based upon the determination that the therapy light beam and/or the secondary light beam is operating during the collecting optical data. The method may also include using the selected data, determining a characteristic of the target.

In an example, a system for determining a characteristic of a target at a target location of a patient may include an optical detector, which may be configured to collect optical datasets from the target location. The system may also include processor circuitry, which may be configured to receive the optical datasets from the optical detector. The processor circuitry may be configured to determine whether to perform analysis on the received optical datasets based at least in part on whether a therapy light beam is operating during collecting of the optical datasets. The analysis may be used to determine a characteristic of the target.

### BRIEF DESCRIPTION OF THE DRAWINGS

In the drawings, which may not be drawn to scale, like numerals may describe substantially similar components throughout one or more of the views. Like numerals having different letter suffixes may represent different instances of substantially similar components. The drawings illustrate generally, by way of example, but not by way of limitation.

FIG. 1 is a drawing of an example of portions of a system for selecting optical data and portions of an environment in which the system for selecting optical data can be used.

FIG. 2 is timing diagram showing an example of operating portions of a system for selecting optical data.

FIG. 3 is timing diagram showing an example of operating portions of a system for selecting optical data.

FIG. 4 is timing diagram showing an example of operating portions of a system for selecting optical data.

FIG. 5 is timing diagram showing an example of operating portions of a system for selecting optical data.

FIG. 6 is timing diagram showing an example of operating portions of a system for selecting optical data.

FIG. 7 is a flow chart showing an example of a method for operating portions of a system for selecting optical data.

FIG. 8 is a block diagram showing an example of a machine that may be used to implement one or more portions of this disclosure.

FIG. 9 is a schematic diagram showing an example of a portion of a computer-based clinical decision support system (CDSS).

### DETAILED DESCRIPTION

This document describes, among other things, a system for selecting optical data that is collected when one or more beams are in a desired operating state.

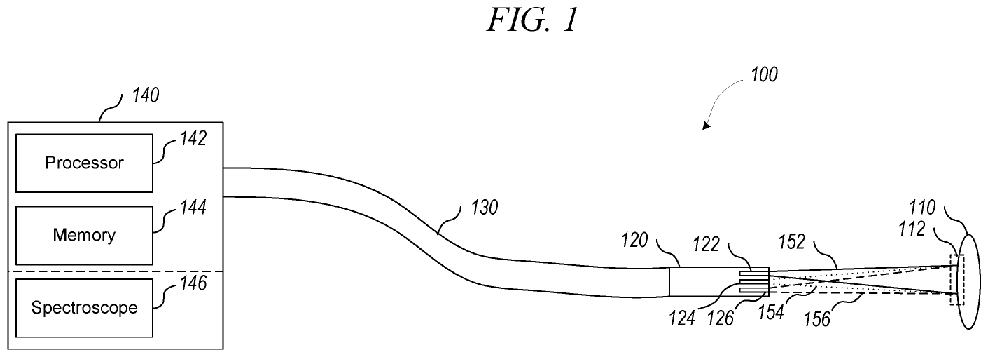

FIG. 1 is a drawing of an example of portions of a system 100 for selecting optical data and portions of an environment in which the system 100 can be used. In the example of FIG. 1, the system 100 can include an endoscope body 130, an endoscope insertion tip 120, and an endoscope controller 140. The environment may include a target 110 including a target location 112.

The endoscope insertion tip 120 may include a therapy beam emitter 122, a secondary beam emitter 124, and an optical detector 126. The therapy beam emitter 122, the secondary beam emitter 124, and the optical detector 126 may be one or more of directed, aimed, or pointed at the target location 112 of the target 110. The therapy beam emitter 122 may have one or more of a focus or field of view that includes a portion of the target location 112, all of the target location 112, or more than all of the target location 112. The secondary beam emitter 124 may have one or more of a focus or field of view that includes a portion of the target location 112, all of the target location 112, or more than all of the target location 112. The optical detector 126 may have one or more of a focus or field of view that includes a portion of the target location 112, all of the target location 112, or more than all of the target location 112. The one or more of a focus or field of view for the therapy beam emitter 122, secondary beam emitter 124, and optical detector 126 may not overlap, may partially overlap, may completely overlap, or any combination thereof.

The therapy beam emitter 122 may be laser, such as may include an infrared laser. The therapy beam emitter 122 may emit a therapy beam 152. The therapy beam 152 may be used in a medical procedure or surgery, such as may be used to ablate, coagulate, vaporize, or fragment a target location 112. In an example, the therapy beam emitter 122 may be a variable power pulsing laser in the infrared spectrum with a wavelength of approximately 2100 nanometers (nm). The therapy beam emitter 122 may generate the therapy beam 152 locally, or the therapy beam 152 may be generated remotely, such as in the endoscope controller 140. The therapy beam 152 may be carried through the endoscope body 130 to the therapy beam emitter 122, such as may include using an optical fiber. The therapy beam 152 may be concentrated in a small region or area or may be a wide beam. The therapy beam 152 may have an adjustable power level (e.g., by adjusting an intensity of the pulses). The therapy beam 152 may have an adjustable average power level such as can be adjusted using pulse width modulation (PWM) to adjust a duty cycle of the therapy beam 152.

The secondary beam emitter 124 may emit a secondary beam 154. The secondary beam emitter 124 may generate the secondary beam 154 locally, or the secondary beam 154 may be generated remotely, such as in the endoscope controller 140. The secondary beam 154 may have an adjustable power level. This may adjust the average power of the secondary beam 154 by using PWM to adjust the duty cycle. The secondary beam 154 may be carried through the endoscope body 130 to the secondary beam emitter 124, such as may include using an optical fiber.

The secondary beam 154 may include a visible aiming beam to allow an operator of the system 100 to visualize where the therapy beam 152 is directed. The visible aiming beam may include a visible aiming laser beam. This may include a green visible laser beam with a wavelength of approximately 500 nm. The visible aiming beam may be generated by a laser diode. The visible aiming beam may be directed to a region next to the region the therapy beam 152 is directed, or may be directed at the same region as the therapy beam 152. The visible aiming beam may be concentrated in a small region or area, or may be a wide beam. An intensity of the visible aiming beam may be adjusted. This may include one or more of adjustment by the operator or automatic adjustment by the endoscope controller 140. In an example, the intensity of the visible aiming beam may be decreased when the endoscope insertion tip 120 is in water or another liquid as compared to when the endoscope insertion tip 120 is in a gas.

The secondary beam emitter 124 may additionally or alternatively include a visible light source to emit a secondary beam 154 in the form of a visible light beam. The visible light beam may allow an operator to see the environment that the endoscope insertion tip 120 is in. This may include seeing the target 110. The visible light source may include a xenon light source, a visible light emitting diode (LED) or an array of LEDs. The visible light source may emit a single wavelength of light, or multiple wavelengths. The visible light beam may illuminate a larger area of the target 110 as opposed to a small area or point. The visible light beam may also illuminate an area around the target 110.

In an example, there may be a visible aiming beam emitter (e.g., green laser diode) and a visible light source emitter (e.g., LED(s) or xenon light source). There may be a visible aiming beam and a visible light beam.

The optical detector 126 may be configured to collect or obtain optical data from the target location 112. The optical detector 126 may have a field of view 156. The optical detector 126 may collect optical data including one or more of overall intensity, the intensity at various spectra, images, and/or other optical data. The optical data collected by the optical detector 126 may be able to provide information about a characteristic of the target 110. In an example the optical detector 126 may not be located in the endoscope insertion tip 120, but may receive transmitted light from the endoscope insertion tip 120, such as may include transmission using an optical fiber.

The target 110 may include a target on or within a patient. The target 110 may include one or more of a tumor or calculi. The target 110 may be accessed by using an endoscope, such as may be used in endoscopic procedures or surgery.

The endoscope body 130 may connect the endoscope controller 140 to the endoscope insertion tip 120. The endoscope body 130 may be rigid or may be flexible in one or more regions. The endoscope body 130 may include channels or instruments or other features to transmit signals, beams, and fluids to and from the endoscope insertion tip 120. The endoscope body 130 may contain one or more lumens or other mechanical structures for transmitting one or more of instruments, tissue, or fluid to and from the endoscope insertion tip 120. The endoscope body 130 may be included in one or more of an endoscope, laparoscope, ureteroscope, arthroscope, or other instrument used for accessing a target location 112 inside a patient.

The endoscope controller 140 may include a processor 142, a memory 144, and a spectroscope 146. The endoscope controller 140 may be connected to other equipment, such as may include a therapy beam generator or a secondary beam generator. The therapy beam generator or secondary beam generator may be communicatively coupled with the endoscope controller 140. The endoscope controller 140 may be configured to control one or more of the therapy beam generator or secondary beam generator. The processor 142 may include circuitry that can be configured to process data from the optical detector 126. The processor 142 may be configured to select data from the optical detector 126 for further processing, such as may include selecting a subset of the data from the optical detector 126. The processor 142 may select optical data using at least one criterion. The memory 144 may include hardware and/or software that can be configured to store and retrieve data. The spectroscope 146 may include circuitry and optics that can be configured to perform spectroscopic analysis on the data from the optical detector 126. In an example, the spectroscope 146 may be separate from the endoscope controller 140, such as may include the spectroscope 146 operating on a separate system and/or device. The spectroscope 146 may be communicatively coupled to the endoscope controller 140, such as may allow the endoscope controller 140 to one or more of interface with the spectroscope 146 or control the spectroscope 146.

The system 100 may include one or more feedback systems for informing or alerting an operator. The operator may include a physician. The alert systems may include at least one of a visible screen, a speaker or other noise generation device, or a touch or haptic alert system. The visible screen may include a color or black-and-white screen, and may display text, figures, symbols, or other visual information. At least a portion of the screen may flash to gain the operators attention. The speaker or other noise generation device may be configured to alert the operator audibly, such as may allow the operator to be alerted when the operator is not looking at the visible screen. The touch or haptic alert system may send a touch signal to a portion of the system 100 that is commonly touched by an operator, such as may include controls or input systems. The touch or haptic alert may vibrate, pulse, or change the shape of the portion of the system touched by the operator.

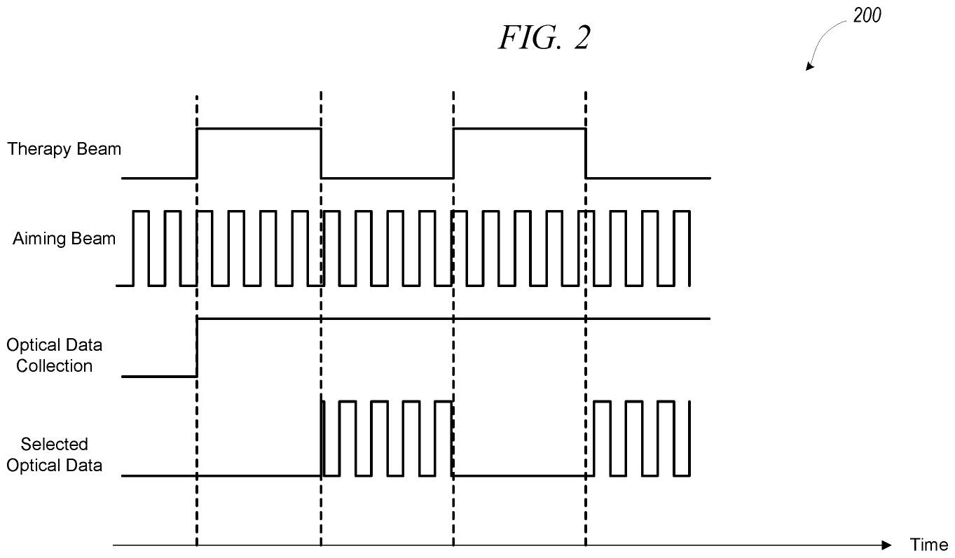

FIG. 2 is timing diagram 200 showing an example of operating portions of a system 100 for selecting optical data. In the example of FIG. 2, the therapy beam 152 may be pulsed. The therapy beam 152 may be pulsed to one or more of limit an average power output, limit a temperature level in one or more of the target 110 of another region, or allow for optical data collection without the therapy beam 152 operating. The secondary beam 154 may be a visible aiming beam. The visible aiming beam may be pulsed. The visible aiming beam may be pulsed to one or more of limit an average power output, such as may provide a desired brightness, or to allow for optical data collection with or without the visible aiming beam operating.

Optical data collection may be continuous or intermittent. Optical data collection may initially be off, and then may be started to run continuously for a period of time (or, in some embodiments, until the end of a medical procedure). The optical data collection may include or consist of analog or digital collection or sampling. The optical data that is collected may be of a consistent format, such as a series of photos or measures of intensity in time. The optical data collected may be of a varying format, such as may include sampling intensity at a variety of frequencies or scanning across a variety of regions of the target 110.

The optical data collection may be conducted without requiring reference to the operation of one or more of the therapy beam 152 or the secondary beam 154. This may allow for the collection/detection of optical data without requiring the synchronization of two or more systems. For example, a therapy beam generator may operate independently of the endoscope controller 140, and there may not be a communication connection between the therapy beam generator system and the endoscope controller 140 or there may be a connection with one or more of a limited bandwidth or variable latency, such as may make synchronization difficult. The collection of optical data without reference to the operating conditions of the system 100 may help in one or more of more accurate optical data, such as may be due to steady state operation, or lower processing strain on the processor 142, such as may be due to not having to sequence the collection or optical data. In an example, the collection of optical data without reference to the operating conditions of the system 100 may allow for a lower precision in the timing of the therapy beam 152, the secondary beam 154, and the collection of optical data.

The endoscope controller 140 may select at least a portion of the received optical data for analysis, such as by using the processor 142. It may be desirable to select for analysis optical data that was collected one or more of while the therapy beam 152 was off or while the therapy beam 152 was operating. The therapy beam 152 may distort or otherwise affect the optical data collected by the optical detector 126, such as may include distorting the spectral components of optical data received from the target 110. The therapy beam 152 may make it more difficult for the endoscope controller 140 to determine a characteristic of the target 110. Therefore, in one embodiment, the target characteristic may be determined based on optical data collected only when the therapy beam 152 is off. In addition, it may be desirable to select for analysis optical data that was collected one or more of while the visible aiming beam was off or while the visible aiming beam was operating. Similar to the therapy beam 152, the visible aiming beam may distort the optical data collected by the optical detector 126, such as may include distorting the spectral components of the optical data received from the target 110. The visible aiming beam may make it more difficult for the endoscope controller 140 to determine a characteristic of the target 110. It may be desirable to select for analysis optical data that was collected while the therapy beam was off and/or the visible aiming beam was off. The optical data that was collected while the visible aiming beam is off may have one or more useful properties, such as may include a more uniform spectral content. Alternatively, the optical data that was collected while the visible aiming beam is on may result in providing a greater illumination level at a given spectral frequency, such as may allow for distinction between various types of targets.

The endoscope controller 140 may one or more of store the selected optical data or use the selected optical data in further operations, such as may include determining a composition or other characteristic of the target 110. Various approaches for determining a characteristic of a target are described in McLoughlin, et al. U.S. Utility Patent Application Serial Number 2021/0038306 entitled “LASER CONTROL USING A SPECTROMETER” filed on Aug. 4, 2020 (Attorney Docket No. 5409.181US1), which is hereby incorporated by reference herein in its entirety. For example, the endoscope controller 140 may determine a composition or other characteristic of the target 110 using a spectral signature indicative of one or more of an absorption spectrum or an emission spectrum of one or more elements or chemical compounds within the target. Alternatively or in addition, the endoscope controller 140 may determine a composition or other characteristic of the target 110 using a system employing machine learning. The machine learning system, such as may include a neural network, may be trained on a series of training data sets to learn to classify targets based on optical data collected from training targets. The endoscope controller 140 may one or more of discard or not store the optical data that is not selected. This may help lower a burden on the endoscope controller 140.

The endoscope controller 140 may collect dark spectra (e.g., background noise). The dark spectra may exist in part due to a temperature of one or more of the target 110 or the target location 112. For example, the dark spectra may be radiation that is characteristic of temperature (i.e., black-body radiation). The dark spectra may be collected using one or more of the optical detector 126 or another sensor. The dark spectra may be sampled with another sensor at the same time the optical detector 126 is collecting the optical data for processing. For example, the dark spectra may be sampled at a location that is not exposed to the visible illumination beam. The dark spectra may be sampled at the same location as the optical data is collected, but may be collected when the visible illumination beam is off. The endoscope controller 140 may subtract a portion or all of the dark spectra from the optical data, such as may provide filtered optical data. The filtered optical data may help with one or more of more accurate determinations about the target 110 or different determinations about the target 110. In an example, the endoscope controller 140 may use a temperature value, such as may be measured by the system 100, to determine a dark spectra. In an example, the endoscope controller 140 may use a temperature value and a dark spectra measurement in determining the dark spectra.

The state of the therapy beam 152 may be determined by the endoscope controller 140 by receiving a signal from the therapy beam emitter 122 or the therapy beam generator as to whether the therapy beam 152 was operating. In an example, the endoscope controller 140 may determine if the therapy beam 152 is operating one or more of before or during the collection of optical data. For example, the therapy beam emitter 122 (or the therapy beam generator) may send a timing or control signal indicating times in the future that the therapy beam 152 will be operating. Additionally or alternatively, the state of the therapy beam 152 may be determined by analyzing the optical data collected by the optical detector 126. In an example the optical data may be analyzed for an indication that the therapy beam 152 was operating, such as may include one or more of indicia of a spectral component of the therapy beam 152 or indicia of a spectral component emitted by the target 110 in response to the therapy beam 152.

The state of the visible aiming beam may be determined by receiving a timing or control signal from the secondary beam emitter 124 or the visible aiming beam generator as to whether the visible aiming beam was operating. Additionally or alternatively, the state of the visible aiming beam may be determined by analyzing the optical data collected by the optical detector 126. In an example, the optical data may be analyzed for an indication that the visible illumination beam was operating, such as may include one or more of indicia of a spectral component of the visible aiming beam or indicia of a spectral component emitted by the target 110 in response to the visible aiming beam. In an example, an intensity of the visible aiming beam may be adjusted to allow for the collection of optical data with one or more properties, such as may include allowing for collection of optical data in a form (e.g., having a sufficient intensity) that can be used to make a determination about the target 110.

In addition, the optical dataset may include or represent a spectral scan, such as may include scanning a range of frequencies and recording a magnitude of optical phenomena at one or more of the scanned frequencies. The optical datasets may be collected recurrently, such as may include recurrently at a substantially constant interval.

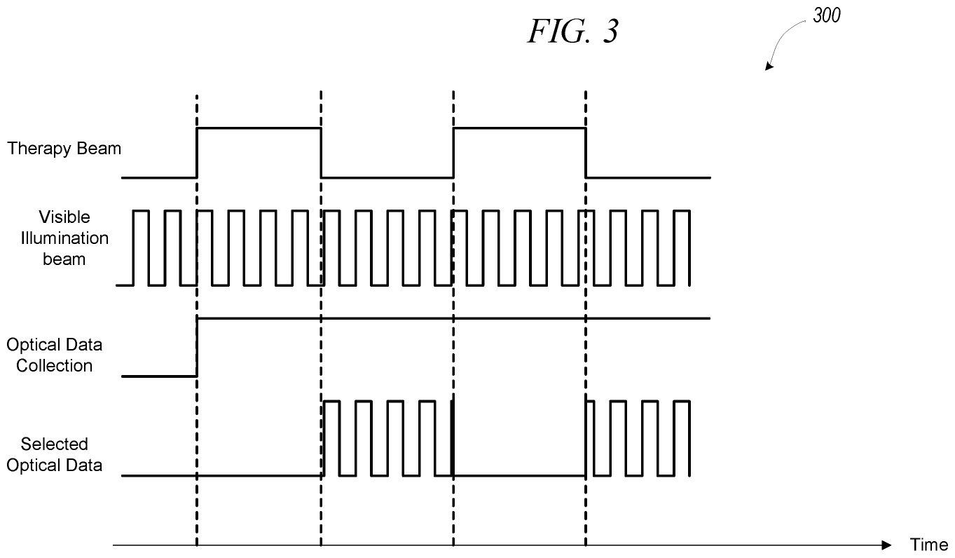

FIG. 3 is timing diagram 300 showing an example of operating portions of a system 100 for selecting optical data. In the example of FIG. 3, the therapy beam 152 may be pulsed. For example, the therapy beam 152 may be pulsed to one or more of limit an average power output, limit a temperature level in one or more of the target 110 of another region, or allow for optical data collection without the therapy beam 152 operating. The secondary beam 154 may be a visible illumination beam. The visible illumination beam may be pulsed. The visible illumination beam may be pulsed to limit an average power output, such as may allow for a desired illumination level.

Optical data collection may be continuous or intermittent. Optical data collection may initially be off, and then may be started to run continuously for a period of time. The optical data collection may include or consist of analog or digital collection or sampling. The optical data that is collected may be of a consistent format, such as a series of images or measures of intensity in time. The optical data collected may be of a varying format, such as may include sampling intensity at a variety of frequencies or scanning across a variety of regions of the target 110.

The endoscope controller 140 may select at least a portion of the received optical data for analysis, such as by using the processor 142. It may be desirable to select optical data for analysis that was collected one or more of while the therapy beam 152 was off or while the therapy beam 152 was operating. It may be desirable to select optical data for analysis that was collected one or more of while the visible illumination beam was off or while the visible illumination beam was operating. The visible illumination beam may provide an illumination level that the optical detector 126 uses to collect optical data. When the visible illumination level beam is off, the optical detector 126 may collect data that is one or more of inaccurate or less accurate than when the visible illumination beam is operating. It may be desirable to select optical data for analysis that was collected while the therapy beam was off and the visible illumination beam was operating.

The state of the visible illumination beam may be determined by receiving a timing or control signal from the secondary beam emitter 124 or the visible illumination beam generator as to whether the visible illumination beam was operating. Additionally or alternatively, the state of the visible illumination beam may be determined by analyzing the optical data collected by the optical detector 126. In an example the optical data may be analyzed for an indication that the visible illumination beam was operating, such as may include one or more of indicia of a spectral component of the visible illumination beam, or indicia of a magnitude or visible light emitted by the target 110. In addition, the optical dataset may include or represent a spectral scan, such as may include scanning a range of frequencies and recording a magnitude of optical phenomena at one or more of the scanned frequencies. The optical datasets may be collected recurrently, such as may include recurrently at a substantially constant interval.

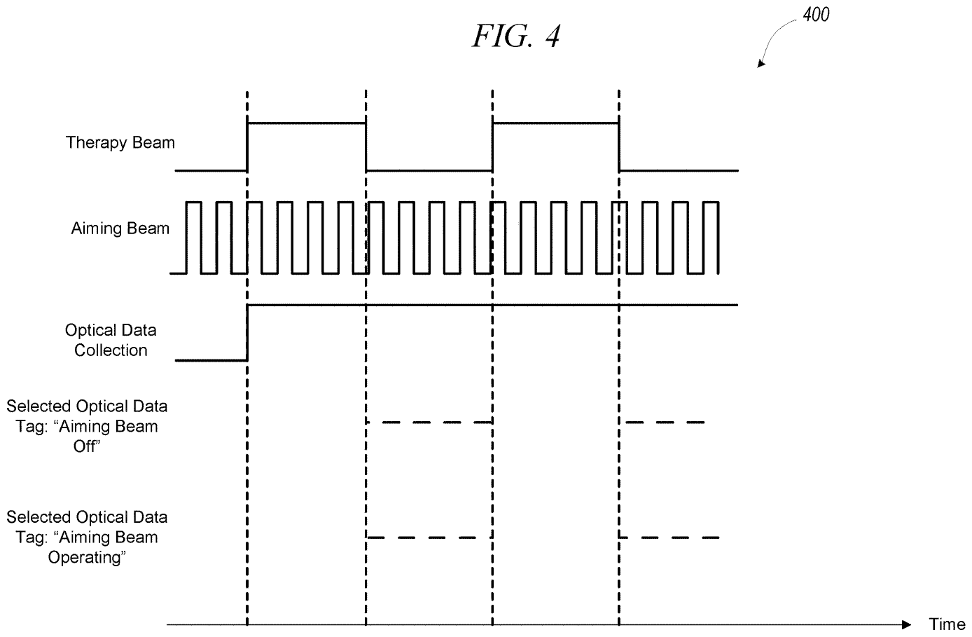

FIG. 4 is timing diagram 400 showing an example of operating portions of a system 100 for selecting optical data. In the example of FIG. 4, the endoscope controller 140 may select optical data that was collected when the therapy beam 152 was off. The endoscope controller 140 may tag the selected data as “aiming beam off” or “aiming beam operating,” such as may be based on whether the aiming beam was off or operating when the selected optical data was collected.

The endoscope controller 140 may one or more of store the selected optical data or use the selected optical data in further operations. The endoscope controller 140 may store or use the selected optical data differently based upon the tag. In an example, the selected optical data tagged “aiming beam off” may be used to make a determination of one or more characteristics of the target 110, while the selected optical data tagged “aiming beam on” may be used to make a determination of one or more different characteristics of the target 110. For example, it may be desirable to use “aiming beam off” data (e.g., so as to reduce the noise level) when making one or more determinations about the target 110, and it may be desirable to use “aiming beam on” data when making one or more other determinations about the target 110 (e.g., so as to have a sufficient intensity of the detected signal). In an example, the endoscope controller 140 may use both the “aiming beam off” data and “aiming beam on” data to make a determination about the target 110. Following the determination, the endoscope controller 140 may select, or allow a user to select, whether to use “aiming beam off” or “aiming beam on” data going forward.

In addition, the optical dataset may include or represent a spectral scan, such as may include scanning a range of frequencies and recording a magnitude of optical phenomena at one or more of the scanned frequencies. The optical datasets may be collected recurrently, such as may include recurrently at a substantially constant interval.

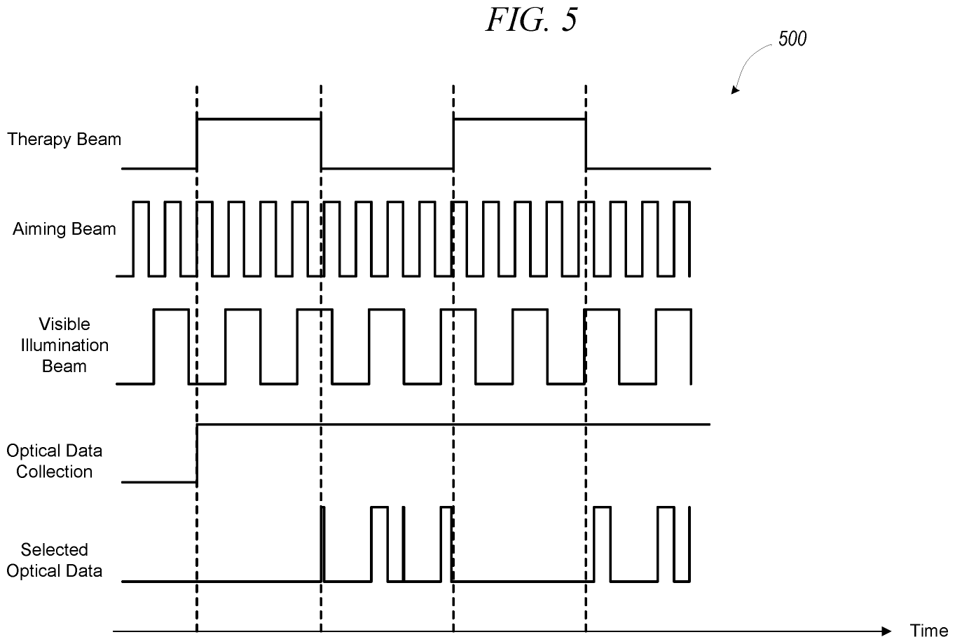

FIG. 5 is timing diagram 500 showing an example of operating portions of a system 100 for selecting optical data. In the example of FIG. 5, there may be a therapy beam 152, and two secondary beams 154—a visible aiming beam and a visible illumination source. The endoscope controller 140 may select optical data based on the operating state of one or more of the therapy beam 152, the visible aiming beam, and the visible illumination beam. The endoscope controller 140 may select data that was collected while the therapy beam 152 was off, the aiming beam was off, and the visible illumination beam was operating. For example, the visible illumination beam may provide a desired level of illumination for the optical detector 126, while the therapy beam 152 and the visible aiming beam may affect the optical data in undesirable ways. The frequency and duty cycle of the beams may be selected to help ensure that optical data with the desired properties will be collected. For example, the endoscope controller 140 may stagger periods or adjust frequencies to provide allow for collection of optical data when each of the beams is in a desired state. In addition, the optical dataset may include or represent a spectral scan, such as may include scanning a range of frequencies and recording a magnitude of optical phenomena at one or more of the scanned frequencies. The optical datasets may be collected recurrently, such as may include recurrently at a substantially constant interval.

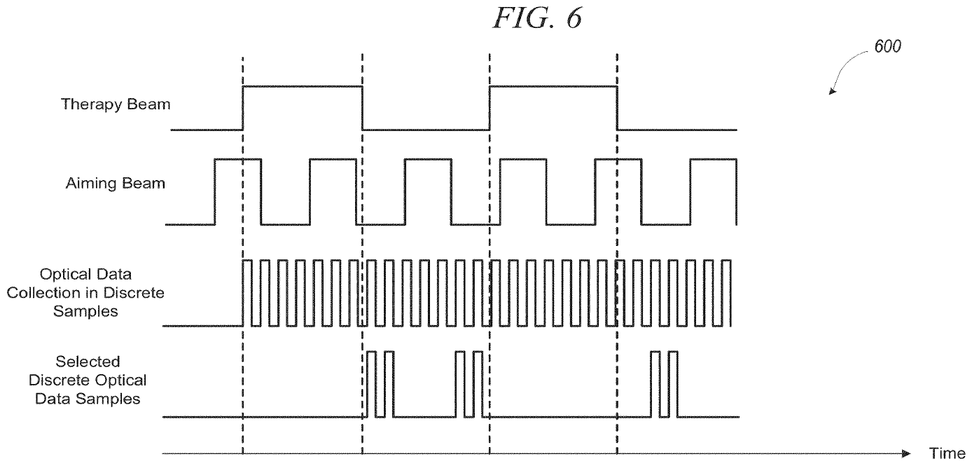

FIG. 6 is timing diagram 600 showing an example of operating portions of a system 100 for selecting optical data. In the example of FIG. 6, the optical detector 126 may collect optical datasets in discrete samples or scans. The optical detector 126 may collect an optical dataset over a period of time, and then may not collect another optical dataset for a period of time. An optical dataset may include or represent a spectral scan, such as may include scanning a range of frequencies and recording a magnitude of optical phenomena at one or more of the scanned frequencies. The optical datasets may be collected recurrently, such as may include recurrently at a substantially constant interval.

The endoscope controller 140 may select a subset of the collected optical datasets for further analysis, such as may include determining a characteristic of the target 110. The endoscope controller 140 may be configured to select or discard entire datasets, and may not be able to, or not configured to, select a subset of a given dataset. For example, an entire dataset may be needed to make a determination about the target 110. The endoscope controller 140 may select datasets that were collected while the therapy beam 152 was off and the aiming beam was off. The endoscope controller 140 may be configured to not select a dataset if the therapy beam 152 and/or the aiming beam were operating for any portion of the dataset collection time. The endoscope controller 140 may select a dataset if the therapy beam 152 and/or the aiming beam were operating for less than a specified portion of the dataset collection time.

The endoscope controller 140 may select or discard entire datasets because an entire data set is desired to make a determination about the target 110, and therefore, it may be desirable to use a dataset that was collected while the various beams are in a constant state. The endoscope controller 140 may discard datasets whose collection time partially overlapped with one or more of the therapy beam 152 operating and/or the aiming beam operating. A partial overlap may make the whole dataset undesirable for further analysis, such as may be due to distorting one or more portions of a spectral collection.

In an example, one or more of the therapy beam, the visible aiming beam, or the visible illumination beam may be sequenced to allow for or result in the collection of optical data or optical datasets that can be selected for further processing. In an example, a characteristic of the therapy beam 152 may be adjusted in response to the analysis of selected optical data, such as may include (1) enable or disable the therapy beam 152, (2) adjust a power setting of the therapy beam 152. In an example, information may be provided to the operator in response to the analysis of selected optical data, such as may include information regarding the target 110. The information regarding the target may include one or more of type of tissue, size of target, distance from the endoscope insertion tip 120 to the target, or any other characteristic.

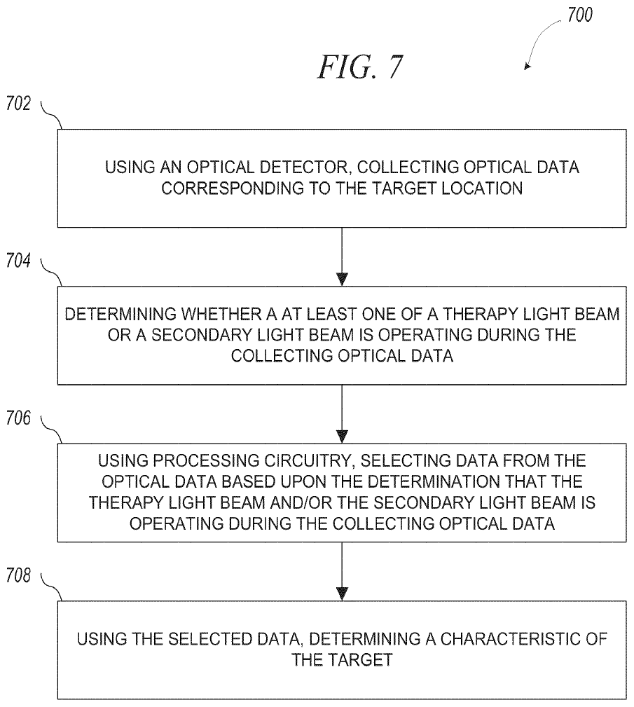

FIG. 7 is a flow chart 700 showing an example of a method for operating portions of a system for selecting optical data. At 702—an optical detector can be used to collect optical data corresponding to the target location. At 704—a determination can be made as to whether at least one of a therapy light beam and/or a secondary light beam is operating during the collection of optical data. At 706—processor circuitry can be used to select data from the optical data based upon the determination that the therapy light beam and/or the secondary light beam is operating during the collection of optical data. At 708—the selected data can be used to determine a characteristic of the target. The shown order of steps is not intended to be a limitation on the order the steps are performed in. In an example, two or more steps may be performed simultaneously or at least partially concurrently.

The optical detector used in 702 may be an optical detector 126 such as may be included in a system 100. The therapy light beam in 704 may be a therapy beam 152 such as may be emitted by a therapy beam emitter 122 in a system 100. The secondary light beam in 704 may be a secondary beam 154 such as may be emitted by a secondary beam emitter 124 in a system 100. In an example, the secondary beam 154 may include one or more of a visible aiming beam or a visible illumination beam. The processor circuitry in 706 may be a processor 142 such as may be included in an endoscope controller 140 in a system 100.

The optical data selected in 706 may be optical data that was collected while the therapy light beam was off and/or while the secondary light beam was off. The optical data selected in 706 may be optical data that was collected while the therapy light beam was off and while the secondary light beam was operating. In an example, there may be two or more secondary beams, such as may include a visible aiming beam and a visible illumination beam. In an example, the processor circuitry in 706 may select optical data based upon the operating state of three or more beams. For example, the processor circuitry in 706 may select optical data that was collected while a therapy beam was off, a visible aiming beam was off, and/or an illumination beam was on.

In an example, the characteristic of the target determined in 708 may be used to adjust a portion of the system 100, such as may include adjusting one or more of the therapy beam emitter 122 and/or the secondary beam emitter 124. For example, the processor 142 may adjust one or more settings of the therapy beam emitter 122 in response to the determined characteristic of the target 110. The processor 142 may adjust one or more of a power, intensity, duty-cycle, or other characteristic of the therapy beam 152, such as to help the therapy beam 152 operate more efficiently for treating the target based upon the determined characteristics of the target 110. The processor 142 may turn off or disable the therapy beam 152 if the target 110 is determined to be an unintended target or unintended region of the target 110.

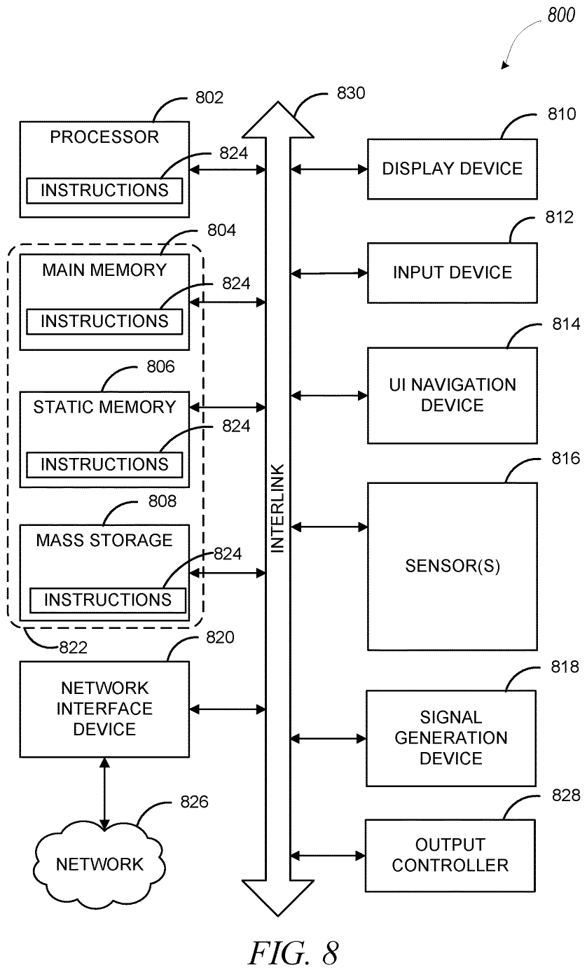

FIG. 8 is a block diagram showing an example of a machine 800 which may be used to implement one or more portions of this disclosure. The machine 800 may operate as a standalone device or may be connected (e.g., networked) to other machines. The machine 800 can be coupled to or connected to the endoscope controller 140 and/or the spectroscope 146 to cause the endoscope controller 140 or the spectroscope 146 to perform one or more of their operations described above. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (Saas), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 800 may include a hardware processor 802 (e.g., a central processing unit (CPU), a graphics processing unit (GPU), a hardware processor core, field programmable gate array (FPGA), or any combination thereof), a main memory 804 and a static memory 806, some or all of which may communicate with each other via an interlink (e.g., bus) 830. The machine 800 may further include a display unit 810, an alphanumeric input device 812 (e.g., a keyboard), and a user interface (UI) navigation device 814 (e.g., a mouse). In an example, the display unit 810, input device 812, and UI navigation device 814 may be a touch screen display. The machine 800 may additionally include a storage device (e.g., drive unit) 808, a signal generation device 818 (e.g., a speaker), a network interface device 820, and one or more sensors 816, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 808 may include a machine readable medium 822 on which is stored one or more sets of data structures or instructions 824 (e.g., software) embodying or used by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 808 may constitute machine readable media.

While the machine readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding, or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 800, and includes digital or analog communications signals or other intangible medium to facilitate communication of such software.

In an example, the hardware processor 802 may be the same as the processor 142. In an example, the 802 may be the same as the endoscope controller 140. In an example, the hardware processor 802 may be separate from the processor 142. In an example, the 802 may be separate from the endoscope controller 140.

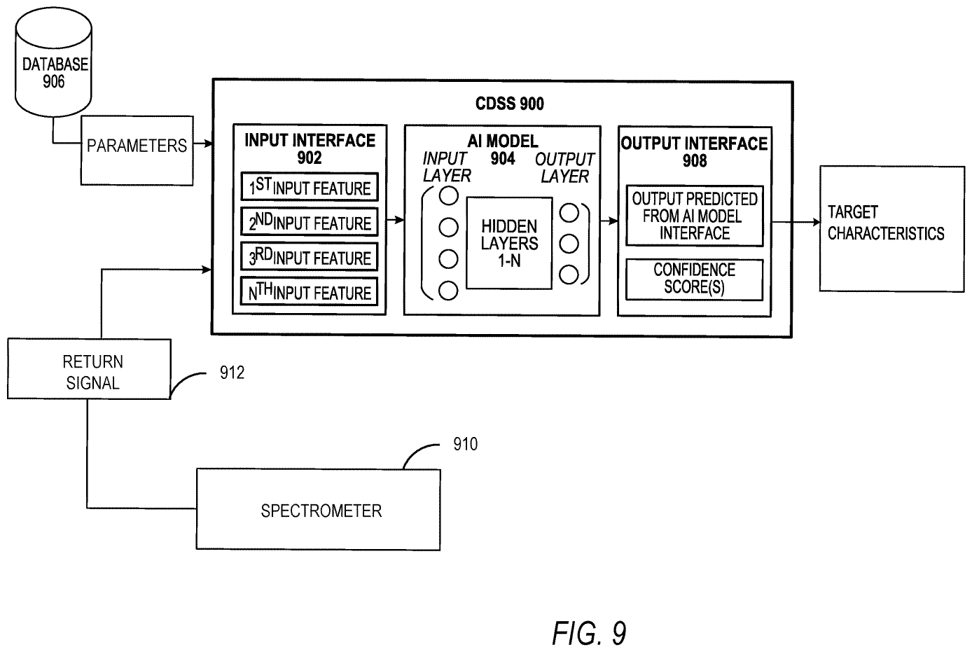

FIG. 9 is a block diagram showing an example of a portion of a computer-based clinical decision support system (CDSS) 900. The CDSS 900 may be configured to determine information or characteristics about a target 110, such as size, composition, hardness, density, or any similar characteristic or information about the target 110 based on spectroscopic or other optical analysis of a signal from the target 110. The CDSS 900 can include an input interface 902 through which parameters such as the size of a surgical fiber, information about the light sources, information about the optical components, and/or information about the scope which are specific to a patient's procedure are provided as input features to an artificial intelligence (AI) model 904, a processor which performs an inference operation in which the parameters are applied to the AI model to generate the determination of the target characteristics, and an output interface 908 through which the determined target characteristics can be communicated to a user, e.g., a clinician.

The input interface 902 may include a direct data link between the CDSS 900 and one or more medical devices that generate at least some of the input features. For example, the input interface 902 may transmit information about the light sources and/or the optical components (e.g., frequency or wavelength of signals from the light sources or aiming beam emission sources, or a rejection frequency of the optical components), or information about a signal returned from the target directly to the CDSS 900 during a therapeutic and/or diagnostic medical procedure. In an example, information about the light sources and/or the optical components, the scope, etc., used during the procedure can be stored in a database 906. Additionally, or alternatively, the input interface 902 may be a classical user interface that facilitates interaction between a user and the CDSS 900. For example, the input interface 902 may facilitate a user interface through which the user may manually enter the information about the surgical fiber, the scope, the optical components, signals to block or allow, etc. Additionally, or alternatively, the input interface 902 may provide the CDSS 900 with access to an electronic patient record or the components being used during the procedure from which one or more input features may be extracted. In any of these cases, the input interface 902 is configured to collect one or more of the following input features in association with one or more of a specific patient, a type of medical procedure, a type of scope, signals that should be rejected during spectroscopic analysis, or the like, on or before a time at which the CDSS 900 is used to assess the input features will take place.

An example of an input feature can include a dimension of the surgical fiber to be used during the procedure.

An example of an input feature can include a type of light or laser source.

An example of an input feature can include the type of scope being used during the procedure.

An example of an input feature can include a wavelength or frequency to be blocked or attenuated.

An example of an input feature can include an amount of time to turn a light or laser source off.

An example of an input feature can include signal information of a return signal 912 received at the spectrometer 910 from the target.

An example of an input feature can include one or more temperature values, such as may include a temperature inside a patient.

Based on one or more of the above input features, the processor may perform an inference operation using the AI model 904 to generate determined characteristics of the target 110 such as the size of the target 110, the composition of the target 110, hardness, density, or any similar characteristic. For example, input interface 902 may deliver the one or more of the input features listed above into an input layer of the AI model 904 which propagates these input features through the AI model 904 to an output layer. The AI model 904 may provide a computer system the ability to perform tasks, without explicitly being programmed, such as by making inferences based on patterns found in the analysis of data. The AI model 904 may explore the study and construction of algorithms (e.g., machine-learning algorithms) that may learn from existing data and make predictions about new data. Such algorithms may operate by building an AI model from example training data in order to make data-driven predictions or decisions expressed as outputs or assessments.

Examples of two modes for machine learning (ML) can include: supervised ML and unsupervised ML. Supervised ML may use prior knowledge (e.g., examples that correlate inputs to outputs or outcomes) to learn the relationships between the inputs and the outputs. The goal of supervised ML may be to learn a function that, given some training data, best approximates the relationship between the training inputs and outputs so that the ML model can implement the same relationships when given inputs to generate the corresponding outputs. Unsupervised ML may be the training of an ML algorithm using information that is neither classified nor labeled and allowing the algorithm to act on that information without guidance. Unsupervised ML may be useful in exploratory analysis because it can automatically identify structure in data.

Tasks for supervised ML can include classification problems and regression problems. Classification problems, also referred to as categorization problems, may aim at classifying items into one of several category values (for example, is this object an apple or an orange?). Regression algorithms may aim at quantifying some items (for example, by providing a score to the value of some input). Some examples of supervised-ML algorithms may include Logistic Regression (LR), Naive-Bayes, Random Forest (RF), neural networks (NN), deep neural networks (DNN), matrix factorization, and Support Vector Machines (SVM).

Some possible tasks for unsupervised ML can include clustering, representation learning, and density estimation. Some examples of unsupervised-ML algorithms are K-means clustering, principal component analysis, and autoencoders.

Another type of ML may be federated learning (also known as collaborative learning) that may train an algorithm across multiple decentralized devices holding local data, without exchanging the data. This approach may stand in contrast to centralized machine-learning techniques where all the local datasets may be uploaded to one server, as well as to decentralized approaches which may assume that local data samples are identically distributed. Federated learning may enable multiple actors to build a common, robust machine learning model without sharing data, which may address considerations such as data privacy, data security, data access rights and access to heterogeneous data.

In some examples, the AI model 904 may be trained continuously or periodically prior to performance of the inference operation by the processor. Then, during the inference operation, the patient specific input features provided to the AI model 904 may be propagated from an input layer, through one or more hidden layers, and ultimately to an output layer that corresponds to the information about the target. For example, when evaluating the spectroscopic analysis of the signal from the target 110, the CDSS 900 may determine one or more characteristics of the target 110.

During and/or subsequent to the inference operation, the information about the target 110 may be communicated to the user via the output interface 908 (e.g., a user interface (UI)) and/or automatically cause a therapeutic laser connected to the processor to perform a desired action. For example, based on the composition of the target the system may cause the therapeutic laser, such as may include the therapy beam 152, to emit energy to ablate the target, adjust the amount of ablation energy, move a portion of the scope, etc.

In an example, the spectrometer 910 may be the same as the spectroscope 146. In an example, the spectrometer 910 may be separate from the spectroscope 146.

### EXAMPLES

Example 1 is a system for selecting and using optical data obtained from a target at a target location of a patient, the system comprising: an optical detector, configured to collect optical data from the target location; and processor circuitry, configured to receive the optical data from the optical detector and to use at least one criterion to select data from the optical data based at least in part on whether a therapy light beam is operating, and to determine a characteristic of the target based on the selected data from the optical data.

In Example 2, the subject matter of Example 1 optionally includes wherein the processor circuitry is configured to select the selected data constituting optical data collected while the therapy light beam is not operating.

In Example 3, the subject matter of any one or more of Examples 1-2 optionally include wherein the processor circuitry is configured to control emission of the therapy light beam by a therapeutic laser in a medical procedure.

In Example 4, the subject matter of any one or more of Examples 1-3 optionally include wherein the processor circuitry is configured to use the at least one criterion to select data from the optical data based at least in part on whether a secondary light beam is operating, and to determine a characteristic of the target based on the selected data from the optical data.

In Example 5, the subject matter of Example 4 optionally includes wherein the secondary light beam includes a visible aiming beam to indicate to an operator where the therapy light beam is directed.

In Example 6, the subject matter of Example 5 optionally includes wherein the processor circuitry is configured to select the selected data constituting optical data collected while the secondary light beam is not operating.

In Example 7, the subject matter of any one or more of Examples 5-6 optionally include wherein the processor circuitry is configured to select the selected data constituting optical data collected while the therapy light beam is not operating, wherein the selected data is processed differently based upon whether the secondary light beam is operating while collecting the optical data.

In Example 8, the subject matter of any one or more of Examples 4-7 optionally include wherein the secondary light beam includes a visible illumination beam.

In Example 9, the subject matter of Example 8 optionally includes wherein the processor circuitry is configured to select the selected data constituting optical data collected while the therapy light beam is not operating and while the secondary light beam is operating.

In Example 10, the subject matter of any one or more of Examples 1-9 optionally include wherein the processor circuitry is configured such that the data that is not selected is at least one of discarded or not stored in memory.

In Example 11, the subject matter of any one or more of Examples 1-10 optionally include wherein the processor circuitry is configured such that the at least one criterion is determined by analyzing the optical data.

In Example 12, the subject matter of any one or more of Examples 1-11 optionally include wherein the processor circuitry is configured to determine the at least one criterion based at least in part on operation of a source of the therapy light beam.

In Example 13, the subject matter of any one or more of Examples 1-12 optionally include wherein the optical detector is included in at least one of a spectroscope or endoscope.

In Example 14, the subject matter of any one or more of Examples 1-13 optionally include wherein the processor circuitry is configured to determine the characteristic of the target at least in part by using spectroscopic analysis of the optical data.

In Example 15, the subject matter of any one or more of Examples 1-14 optionally include ) provide information to an operator.

In Example 16, the subject matter of Example 15 optionally includes wherein provided information includes at least one of visual, audible or haptic feedback.

Example 17 is a computer-implemented method of operating a system for selecting optical data obtained from a target at a target location of a patient, the method comprising: using an optical detector, collecting optical data corresponding to the target location; determining whether at least one of a therapy light beam or a secondary light beam is operating during the collecting optical data; using processor circuitry, selecting data from the optical data based upon the determination that the therapy light beam and/or the secondary light beam is operating during the collecting optical data; and using the selected data, determining a characteristic of the target.

In Example 18, the subject matter of Example 17 optionally includes wherein the selected data corresponds to optical data collected while at least one of the therapy light beam or the secondary light beam is off.

In Example 19, the subject matter of any one or more of Examples 17-18 optionally include wherein the determining whether at least one of the therapy light beam or the secondary light beam is operating during the collecting optical data occurs at least one of before or during the collecting optical data.

Example 20 is a system for determining a characteristic of a target at a target location of a patient, the system comprising: an optical detector, configured to collect optical datasets from the target location; processor circuitry, configured to receive the optical datasets from the optical detector; wherein the processor circuitry is configured to determine whether to perform analysis on the received optical datasets based at least in part on whether a therapy light beam is operating during collecting of the optical datasets; and wherein the analysis is used to determine a characteristic of the target.

In Example 21, the subject matter of Example 20 optionally includes wherein the optical datasets are collected recurrently at a substantially constant interval.

In Example 22, the subject matter of any one or more of Examples 20-21 optionally include wherein the optical detector is a spectroscope and the optical datasets represent spectral scans.

In Example 23, the subject matter of Example 22 optionally includes wherein the therapy light beam is sequenced so that it is not operating during collecting of at least a subset of the optical datasets.

Example 24 is at least one machine-readable medium including instructions that, when executed by processing circuitry, cause the processing circuitry to perform operations to implement of any one or more of Examples 1-23.

Example 25 is an apparatus comprising means to implement of any one or more of Examples 1-23.

Example 26 is a system to implement of any one or more of Examples 1-23.

Example 27 is a method to implement of any one or more of Examples 1-23.

Each of the non-limiting aspects above can stand on its own or can be combined in various permutations or combinations with one or more of the other aspects or other subject matter described in this document.

The above detailed description includes references to the accompanying drawings, which form a part of the detailed description. The drawings show, by way of illustration, specific embodiments in which the invention can be practiced. These embodiments are also referred to generally as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc., are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Such instructions can be read and executed by one or more processors to enable performance of operations comprising a method, for example. The instructions are in any suitable form, such as but not limited to source code, compiled code, interpreted code, executable code, static code, dynamic code, and the like.

Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system for selecting and using optical data obtained from a target at a target location of a patient, the system comprising:
an optical detector, configured to collect optical data from the target location, wherein at least some of the optical data is collected while a therapy light beam is operating; and
processor circuitry, configured to receive the optical data from the optical detector and to use at least one criterion to select data from the optical data based at least in part on whether the therapy light beam is operating and to determine a characteristic of the target based on the selected data from the optical data.

2. The system of claim 1, wherein the processor circuitry is configured to select the selected data constituting optical data collected while the therapy light beam is not operating.

3. The system of claim 1, wherein the processor circuitry is configured to control emission of the therapy light beam by a therapeutic laser in a medical procedure.

4. The system of claim 1, wherein the processor circuitry is configured to use the at least one criterion to select data from the optical data based at least in part on whether a secondary light beam is operating, and to determine a characteristic of the target based on the selected data from the optical data.

5. The system of claim 4, wherein the secondary light beam includes a visible aiming beam to indicate to an operator where the therapy light beam is directed.

6. The system of claim 5, wherein the processor circuitry is configured to select the selected data constituting optical data collected while the secondary light beam is not operating.

7. The system of claim 5, wherein the processor circuitry is configured to select the selected data constituting optical data collected while the therapy light beam is not operating, wherein the selected data is processed differently based upon whether the secondary light beam is operating while collecting the optical data.

8. The system of claim 4, wherein the secondary light beam includes a visible illumination beam.

9. The system of claim 8, wherein the processor circuitry is configured to select the selected data constituting optical data collected while the therapy light beam is not operating and while the secondary light beam is operating.

10. The system of claim 1, wherein the processor circuitry is configured such that the data that is not selected is at least one of discarded or not stored in memory.

11. The system of claim 1, wherein the processor circuitry is configured such that the at least one criterion is determined by analyzing the optical data.

12. The system of claim 1, wherein the processor circuitry is configured to determine the at least one criterion based at least in part on operation of a source of the therapy light beam.

13. The system of claim 1, wherein the optical detector is included in at least one of a spectroscope or endoscope.

14. The system of claim 1, wherein the processor circuitry is configured to determine the characteristic of the target at least in part by using spectroscopic analysis of the optical data.

15. The system of claim 1, wherein the processor circuitry is configured to use the determined characteristic to at least one of (1) enable or disable the therapy light beam, (2) adjust a power setting of the therapy light beam, or (3) provide information to an operator.

16. The system of claim 15, wherein provided information includes at least one of visual, audible or haptic feedback.

17. A computer-implemented method of operating a system for selecting optical data obtained from a target at a target location of a patient, the method comprising:
using an optical detector, collecting optical data corresponding to the target location;
determining whether a monitored one of a therapy light beam or a secondary light beam is operating during the collecting optical data, wherein the monitored one of the therapy light beam or the secondary light beam changes operational states during collecting optical data;
using processor circuitry, selecting data from the optical data based upon the determination that the monitored one of the therapy light beam or the secondary light beam is operating during the collecting optical data; and
using the selected data, determining a characteristic of the target.

18. The method of claim 17, wherein the selected data corresponds to optical data collected while at least one of the therapy light beam or the secondary light beam is off.

19. The method of claim 17, wherein the determining whether at least one of the therapy light beam or the secondary light beam is operating during the collecting optical data occurs at least one of before or during the collecting optical data.

20. A system for determining a characteristic of a target at a target location of a patient, the system comprising:
an optical detector, configured to collect optical datasets from the target location, wherein at least some of the optical datasets are collected while a therapy light beam is operating;
processor circuitry, configured to receive the optical datasets from the optical detector;
wherein the processor circuitry is configured to determine whether to perform analysis on the received optical datasets based at least in part on whether a therapy light beam is operating during collecting of the optical datasets; and
wherein the analysis is used to determine a characteristic of the target.
